(19) United States
(12) Patent Application Publication
(10) Pub. No.: US 2021/0206644 A1
(43) Pub. Date: Jul. 8, 2021

(54) SUBSTITUTED GRAPHANE MATERIAL WITH THREE-DIMENSIONAL STRUCTURE AND PREPARATION METHOD THEREOF

(71) Applicant: Huaqiao University, Xiamen (CN)

(72) Inventors: Guohua CHEN, Xiamen (CN); Songlin CHEN, Xiamen (CN); Xiaomin ZHAO, Xiamen (CN); Danqing CHEN, Xiamen (CN); Lengshen HUANG, Xiamen (CN); Yajing HUANG, Xiamen (CN)

(21) Appl. No.: 17/192,589

(22) Filed: Mar. 4, 2021

Related U.S. Application Data
(63) Continuation of application No. PCT/CN2018/118221, filed on Nov. 29, 2018.

(30) Foreign Application Priority Data
Sep. 6, 2018 (CN) 201811036828.7

(51) Int. Cl. C01B 32/194; C01B 32/192

(52) U.S. Cl. CPC C01B 32/194; B82Y 30/00; C01B 32/192

(57) ABSTRACT

The present disclosure discloses a substituted graphane material with a three-dimensional structure. The substituted graphane material comprises a planar substrate with a plurality of six-membered carbon rings comprising continuous sp³ hybrids, wherein an organic molecular ring is connected to the planar substrate due to a Diels-Alder (D-A) reaction.

### RELATED APPLICATIONS

This application a continuation application of International Patent Application PCT/CN2018/118221, filed on Nov. 29, 2018, which claims priority to Chinese patent application number 201811036828.7, filed Sep. 6, 2018. International Patent Application PCT/CN2018/118221 and Chinese patent application number 201811036828.7 are incorporated herein by reference.

### FIELD OF THE DISCLOSURE

The present disclosure relates to graphene material, and in particular relates to a substituted graphane material with a three-dimensional structure and a preparation method thereof.

### BACKGROUND OF THE DISCLOSURE

Carbon is one of the earliest elements that humans came into contact with and one of the earliest elements that humans used. The radius of a carbon atom is small, the electron configuration is 1s²2s²2p², the inner shell is a spherical 1s² orbit, and the outer shell 2s and 2p orbits are easy to hybridize into bonds, which can form stable single, double and triple bonds. This diverse form of bonding enables the carbon atom to form a variety of structures such as chain, ring, mesh, and other structures.

Carbon materials with different structures may have different properties and thus have different applications. C₆₀ (carbon 60, buckyball), discovered in 1985, is a zero-dimensional nanomaterial fullerene, which has very good photoelectric properties. Carbon nanotube, discovered in 1991, is a one-dimensional nanomaterial, which not only has very good electrical properties, but also has excellent mechanical properties. Graphene, which was discovered in 2004, also has photoelectrical, mechanical, and other characteristics, and is playing a role in various fields. If the conjugated unsaturated double bond on graphene is opened by adding hydrogen atoms, sp² carbon atoms on graphene will be converted to spa hybridization, and graphene will also be converted into graphane. At present, there are reports of predicting the possible characteristics of graphane through theoretical calculations, but so far there is no effective synthesis method.

### BRIEF SUMMARY OF THE DISCLOSURE

The present disclosure provides a substituted graphane material with three-dimensional structure.

The present disclosure further provides a method for preparing the substituted graphane material with three-dimensional structure.

The principle of the present disclosure is carried out using the Diels-Alder reaction (D-A reaction). The D-A reaction is a cycloaddition reaction between a conjugated diene system and an alkene bond or an alkyne bond to obtain a cyclohexene or 1,4-cyclohexadiene ring system. In 1928, German chemists O P H Diels and K. Alder discovered this type of reaction when studying the interaction between butadiene and maleic dianhydride. Olefins and alkynes that interact with conjugated dienes are called dienophiles. Electrophilic substituents of dienophiles and electron-donating substituents of the conjugated dienes have reaction accelerating effects. Its basic reaction is shown in FIG. 1. According to the principle of the D-A reaction, graphene can be regarded as a dienophile, which can undergo D-A reaction with the conjugated dienes. The conjugated dienes can be gradually bonded to a graphene plane and double bonds of the graphene are opened to be bonded to carbon atom of the graphene due to excess organic molecules comprising the conjugated dienes reacting with reduced graphene oxide.

A technical solution of the present disclosure is as follows.

A substituted graphane material with a three-dimensional structure comprises a planar substrate with a plurality of six-membered carbon rings comprising continuous spa hybrids, wherein an organic molecular ring is connected to the planar substrate due to a Diels-Alder (D-A) reaction.

In a preferred embodiment of the present disclosure, the organic molecular ring is a ring merely comprising a single organic molecular or a ring comprising a plurality of continuous organic molecules.

In a preferred embodiment of the present disclosure, the organic molecular ring is distributed on a side of a plane, and the planar substrate is disposed on the plane.

In a preferred embodiment of the present disclosure, the organic molecular ring is distributed on two sides of a plane, and the planar substrate is disposed on the plane (see FIG. 3).

A method for preparing the substituted graphane material according to claim 1, comprising:

(1) mixing graphene material and conjugated diene, heating in inert gas or nitrogen atmosphere to a temperature at which the conjugated diene is configured to form a reflux, and refluxing and reacting for 6-8 days to obtain a material; and

(2) removing impurities of the material with a solvent and drying to remove the solvent such that the substituted graphane material is obtained.

In a preferred embodiment of the present disclosure, the conjugated diene comprises 2-furan methylamine, 2-thienyl methylamine, 2-furan methanol, or 2-thienyl methanol.

In a preferred embodiment of the present disclosure, the solvent comprises one or two of water, toluene, acetone, and chloroform.

In a preferred embodiment of the present disclosure, the inert gas is argon gas.

In a preferred embodiment of the present disclosure, the graphene material is reduced graphene oxide or mechanical stripping graphene.

In a preferred embodiment of the present disclosure, a temperature of the reflux is 80° C.-150° C.

Compared with the existing techniques, the technical solution has the following advantages.

The substituted graphane material prepared by the present disclosure is a new macromolecule. It is a three-dimensional macromolecular structure formed by bonding organic molecules on planar six-membered carbon rings comprising continuous sp³ hybrids. Because the basic structure of the three-dimensional macromolecule is very different from the traditional linear molecular structure, the three-dimensional macromolecule will have many different properties from existing materials and have advantages in many properties. For example, the three-dimensional macromolecules obtained by the reaction between graphene and furan methylamine have good dissolution properties and can be dissolved in organic solvents such as ethanol, toluene, acetone, tetrahydrofuran, chloroform, cyclohexanone, etc., methyl methacrylate, styrene, polypolyacid, epoxy prepolymer, and other polymer monomers. The three-dimensional macromolecule can form a film, form a transparent film on a surface of water, has a certain toughness (see FIG. 4), and so on. The selected diffraction by TEM shows that the prepared product no longer has the original graphene diffraction ring (see FIG. 5), indicating that the regular two-dimensional crystal structure of the original graphene disappears and turns into a nonconjugated structure of graphane. The hydrogen nuclear magnetic resonance spectrum and carbon nuclear magnetic resonance spectrum show that the newly generated methylene and methine groups of sp³C appear on the prepared product (see FIGS. 6 and 7), indicating that furan methylamine is grafted onto the planar six-membered carbon rings comprising the continuous sp³ hybrids by the D-A reaction.

### BRIEF DESCRIPTION OF THE DRAWINGS

FIG. 1 illustrates a schematic diagram of the Diels-Alder (D-A) reaction.

FIG. 2 illustrates a conceptual schematic diagram of a structure of a substituted graphane of the present disclosure.

FIG. 3 illustrates a schematic diagram of a reaction between graphene and conjugated diene (i.e., furan methylamine) of the present disclosure (i.e., RGO is reduced graphene oxides, graphanes are substituted graphanes).

FIG. 4 illustrates a schematic diagram of a film formation process of the substituted graphane of the present disclosure.

FIG. 5a illustrates an electron diffraction diagram of the substituted graphane of the present disclosure (characteristic diagram without lattice structure).

FIG. 5b illustrates a TEM (transmission electron microscopy) diagram of the substituted graphane of the present disclosure.

FIG. 6 illustrates a hydrogen nuclear magnetic resonance spectrum of the substituted graphane of the present disclosure.

FIG. 7 illustrates a carbon nuclear magnetic resonance spectrum of the substituted graphane of the present disclosure.

### DETAILED DESCRIPTION OF THE EMBODIMENTS

The present disclosure will be further described below in combination with the accompanying drawings and embodiments.

### Embodiment 1

1. 2 g 8000 mesh graphite powder, 12 g potassium permanganate, and 100 mL concentrated sulfuric acid (i.e., a weight concentration of the concentrated sulfuric acid was 70%-98%) was weighed.

2. 2 g graphite powder and 100 mL concentrated sulfuric acid were added into a 500 mL beaker, potassium permanganate was added into the 500 mL beaker in an ice bath to obtain a first solution, the first solution was kept in the ice bath for half an hour and was maintained at 35° C. for 2 hours, 150 mL distilled water was then slowly added into the first solution, and hydrogen peroxide was added into the first solution at room temperature (i.e., a temperature of 25° C.-30° C.).

3. The first solution was filtered by suction.

4. The first solution was respectively centrifuged with 5% dilute hydrochloric acid and distilled water.

5. The first solution was freeze-dried to obtain GO (graphene oxide)

6. 0.05 g GO was taken and evenly dispersed in 300 mL deionized water to obtain a second solution by high-frequency ultrasound (i.e., 20 khz-40 kHz), and 15 g VC (ascorbic acid) was taken and dissolved in the second solution.

7. The second solution was evenly dispersed and then was put in an oven at 80° C. for 4 hours.

8. The second solution was cooled to room temperature, filtered, washed by deionized water until residual material was washed away, and was dried in vacuum (i.e., 0.001 atm-0.1 atm) to obtain RGO (reduced graphene oxides).

9. 0.05 g RGO and 4 mL 2-furan methylamine was added into a three-necked flask to obtain a third solution.

10. The third solution was placed in oil bath maintained at 80° C. and was stirred by magnet, air in the three-necked flask was replaced by nitrogen (or inert gas, for example, argon gas) at standard atmospheric pressure (i.e., 1 atm), and the third solution was reacted for 7 days.

11. 20 mL toluene and 100 mL deionized water were added into the third solution, and the deionized water was removed. 100 mL deionized water was added and removed 3 times to obtain a toluene mixture.

12. The toluene mixture was dried (45° C., vacuum pressure (i.e., 0.001 atm-0.1 atm)) for 6 hours and the oven was opened 4 times, and then the toluene mixture was freeze-dried for 48 hours to obtain the sample shown in FIG. 2.

### Embodiment 2

1. 2 g 8000 mesh graphite powder, 12 g potassium permanganate, and 100 mL concentrated sulfuric acid was weighed.

2. 2 g graphite powder and 100 mL concentrated sulfuric acid were added into a 500 mL beaker, potassium permanganate was added into the 500 mL beaker in an ice bath to obtain a first solution, the first solution was kept in the ice bath for half an hour and was maintained at 35° C. for 2 hours, 150 mL distilled water was then slowly added into the first solution, and hydrogen peroxide was added into the first solution at room temperature.

3. The first solution was filtered by suction.

4. The first solution was respectively centrifuged with 5% dilute hydrochloric acid and distilled water.

5. The first solution was freeze-dried to obtain GO (graphene oxide)

6. 0.05 g GO was taken and evenly dispersed in 300 mL deionized water to obtain a second solution by high-frequency ultrasound, and 15 g VC (ascorbic acid) was taken and dissolved in the second solution.

7. The second solution was evenly dispersed and then was put in an oven at 80° C. for 4 hours.

8. The second solution was cooled to room temperature, filtered, washed by deionized water until residual material was washed away, and was dried in vacuum to obtain RGO.

9. 0.05 g RGO and 4 mL 2-furan methanol was added into a three-necked flask to obtain a third solution.

10. The third solution was placed in oil bath maintained at 130° C. and was stirred by magnet, air in the three-necked flask was replaced by nitrogen at standard atmospheric pressure, and the third solution was reacted for 7 days.

11. 20 mL toluene and 100 mL deionized water were added into the third solution, and the deionized water was removed. 100 mL deionized water was added and removed 3 times to obtain a toluene mixture.

12. The toluene mixture was dried (45° C., vacuum pressure) for 6 hours and the oven was opened 4 times, and then the toluene mixture was freeze-dried for 48 hours to obtain the sample shown in FIG. 2.

### Embodiment 3

1. 2 g 8000 mesh graphite powder, 12 g potassium permanganate, and 100 mL concentrated sulfuric acid was weighed.

2. 2 g graphite powder and 100 mL concentrated sulfuric acid were added into a 500 mL beaker, potassium permanganate was added into the 500 mL beaker in an ice bath to obtain a first solution, the first solution was kept in the ice bath for half an hour and was maintained at 35° C. for 2 hours, 150 mL distilled water was then slowly added into the first solution, and hydrogen peroxide was added into the first solution at room temperature.

3. The first solution was filtered by suction.

4. The first solution was respectively centrifuged with 5% dilute hydrochloric acid and distilled water.

5. The first solution was freeze-dried to obtain GO (graphene oxide)

6. 0.05 g GO was taken and evenly dispersed in 300 mL deionized water to obtain a second solution by high-frequency ultrasound, and 15 g VC (ascorbic acid) was taken and dissolved in the second solution.

7. The second solution was evenly dispersed and then was put in an oven at 80° C. for 4 hours.

8. The second solution was cooled to room temperature, filtered, washed by deionized water until residual material was washed away, and was dried in vacuum to obtain RGO.

9. 0.05 g RGO and 4 mL 2-thiophene methylamine was added into a three-necked flask to obtain a third solution.

10. The third solution was placed in oil bath maintained at 80° C. and was stirred by magnet, air in the three-necked flask was replaced by nitrogen at standard atmospheric pressure, and the third solution was reacted for 7 days.

11. 20 mL toluene and 100 mL deionized water were added into the third solution, and the deionized water was removed. 100 mL deionized water was added and removed 3 times to obtain a toluene mixture.

12. The toluene mixture was dried (45° C., vacuum pressure) for 6 hours and the oven was opened 4 times, and then the toluene mixture was freeze-dried for 48 hours to obtain the sample shown in FIG. 2.

### Embodiment 4

1. 2 g 8000 mesh graphite powder, 12 g potassium permanganate, and 100 mL concentrated sulfuric acid was weighed.

2. 2 g graphite powder and 100 mL concentrated sulfuric acid were added into a 500 mL beaker, potassium permanganate was added into the 500 mL beaker in an ice bath to obtain a first solution, the first solution was kept in the ice bath for half an hour and was maintained at 35° C. for 2 hours, 150 mL distilled water was then slowly added into the first solution, and hydrogen peroxide was added into the first solution at room temperature.

3. The first solution was filtered by suction.

4. The first solution was respectively centrifuged with 5% dilute hydrochloric acid and distilled water.

5. The first solution was freeze-dried to obtain GO (graphene oxide)

6. 0.05 g GO was taken and evenly dispersed in 300 mL deionized water to obtain a second solution by high-frequency ultrasound, and 15 g VC (ascorbic acid) was taken and dissolved in the second solution.

7. The second solution was evenly dispersed and then was put in an oven at 80° C. for 4 hours.

8. The second solution was cooled to room temperature, filtered, washed by deionized water until residual material was washed away, and was dried in vacuum to obtain RGO.

9. 0.05 g RGO and 4 mL 2-thiophene methanol was added into a three-necked flask to obtain a third solution.

10. The third solution was placed in oil bath maintained at 150° C. and was stirred by magnet, air in the three-necked flask was replaced by nitrogen at standard atmospheric pressure, and the third solution was reacted for 7 days.

11. 20 mL toluene and 100 mL deionized water were added into the third solution, and the deionized water was removed. 100 mL deionized water was added and removed 3 times to obtain a toluene mixture.

12. The toluene mixture was dried (45° C., vacuum pressure) for 6 hours and the oven was opened 4 times, and then the toluene mixture was freeze-dried for 48 hours to obtain the sample shown in FIG. 2.

### Embodiment 5

1. 0.05 g G2 (industrial graphene) and 4 mL 2-furan methylamine was added into a three-necked flask to obtain a third solution.

2. The third solution was placed in oil bath maintained at 80° C. and was stirred by magnet, air in the three-necked flask was replaced by nitrogen at standard atmospheric pressure, and the third solution was reacted for 7 days.

3. 20 mL toluene and 100 mL deionized water were added into the third solution, and the deionized water was removed. 100 mL deionized water was added and removed 3 times to obtain a toluene mixture.

4. The toluene mixture was dried (45° C., vacuum pressure) for 6 hours and the oven was opened 4 times, and then the toluene mixture was freeze-dried for 48 hours to obtain the sample shown in FIG. 2.

### Embodiment 6

1. 0.05 g G2 (industrial graphene) and 4 mL 2-furan methanol was added into a three-necked flask to obtain a third solution.

2. The third solution was placed in oil bath maintained at 130° C. and was stirred by magnet, air in the three-necked flask was replaced by nitrogen at standard atmospheric pressure, and the third solution was reacted for 7 days.

3. 20 mL toluene and 100 mL deionized water were added into the third solution, and the deionized water was removed. 100 mL deionized water was added and removed 3 times to obtain a toluene mixture.

4. The toluene mixture was dried (45° C., vacuum pressure) for 6 hours and the oven was opened 4 times, and then the toluene mixture was freeze-dried for 48 hours to obtain the sample shown in FIG. 2.

### Embodiment 7

1. 0.05 g G2 (industrial graphene) and 4 mL 2-thiophene methylamine was added into a three-necked flask to obtain a third solution.

2. The third solution was placed in oil bath maintained at 80° C. and was stirred by magnet, air in the three-necked flask was replaced by nitrogen at standard atmospheric pressure, and the third solution was reacted for 7 days.

3. 20 mL toluene and 100 mL deionized water were added into the third solution, and the deionized water was removed. 100 mL deionized water was added and removed 3 times to obtain a toluene mixture.

4. The toluene mixture was dried (45° C., vacuum pressure) for 6 hours and the oven was opened 4 times, and then the toluene mixture was freeze-dried for 48 hours to obtain the sample shown in FIG. 2.

### Embodiment 8

1. 0.05 g G2 (industrial graphene) and 4 mL 2-thiophene methanol was added into a three-necked flask to obtain a third solution.

2. The third solution was placed in oil bath maintained at 150° C. and was stirred by magnet, air in the three-necked flask was replaced by nitrogen at standard atmospheric pressure, and the third solution was reacted for 7 days.

3. 20 mL toluene and 100 mL deionized water were added into the third solution, and the deionized water was removed. 100 mL deionized water was added and removed 3 times to obtain a toluene mixture.

4. The toluene mixture was dried (45° C., vacuum pressure) for 6 hours and the oven was opened 4 times, and then the toluene mixture was freeze-dried for 48 hours to obtain the sample shown in FIG. 2.

### Embodiment 9

1. 0.5 g 8000 mesh flake graphite and 40 mL NMP (N-methylpyrrolidone) were added into a ball mill tank comprising zirconia milling balls and was planetary ball milled for 48 hours to obtain a mixture.

2. The mixture was filtered and washed by suction 10 times by distilled water and ethanol and was dried at 80° C. to obtain graphene.

3. 0.05 g industrial graphene and 4 mL 2-furan methylamine was added into a three-necked flask to obtain a third solution.

4. The third solution was placed in oil bath maintained at 80° C. and was stirred by magnet, air in the three-necked flask was replaced by nitrogen at standard atmospheric pressure, and the third solution was reacted for 7 days.

5. 20 mL toluene and 100 mL deionized water were added into the third solution, and the deionized water was removed. 100 mL deionized water was added and removed 3 times to obtain a toluene mixture.

6. The toluene mixture was dried (45° C., vacuum pressure) for 6 hours and the oven was opened 4 times, and then the toluene mixture was freeze-dried for 48 hours to obtain the sample shown in FIG. 2.

### Embodiment 10

1. 0.5 g 8000 mesh flake graphite and 40 mL NMP (N-methylpyrrolidone) were added into a ball mill tank comprising zirconia milling balls and was planetary ball milled for 48 hours to obtain a mixture.

2. The mixture was filtered and washed by suction 10 times by distilled water and ethanol and was dried at 80° C. to obtain graphene.

3. 0.05 g industrial graphene and 4 mL 2-furan methanol was added into a three-necked flask to obtain a third solution.

4. The third solution was placed in oil bath maintained at 130° C. and was stirred by magnet, air in the three-necked flask was replaced by nitrogen at standard atmospheric pressure, and the third solution was reacted for 7 days.

5. 20 mL toluene and 100 mL deionized water were added into the third solution, and the deionized water was removed. 100 mL deionized water was added and removed 3 times to obtain a toluene mixture.

6. The mixture was dried (45° C., vacuum pressure) for 6 hours and the oven was opened 4 times, and then the toluene mixture was freeze-dried for 48 hours to obtain the sample shown in FIG. 2.

### Embodiment 11

1. 0.5 g 8000 mesh flake graphite and 40 mL NMP (N-methylpyrrolidone) were added into a ball mill tank comprising zirconia milling balls and was planetary ball milled for 48 hours to obtain a mixture.

2. The mixture was filtered and washed by suction 10 times by distilled water and ethanol and was dried at 80° C. to obtain graphene.

3. 0.05 g industrial graphene and 4 mL 2-thiophene methylamine was added into a three-necked flask to obtain a third solution.

4. The third solution was placed in oil bath maintained at 80° C. and was stirred by magnet, air in the three-necked flask was replaced by nitrogen at standard atmospheric pressure, and the third solution was reacted for 7 days.

5. 20 mL toluene and 100 mL deionized water were added into the third solution, and the deionized water was removed. 100 mL deionized water was added and removed 3 times to obtain a toluene mixture.

6. The toluene mixture was dried (45° C., vacuum pressure) for 6 hours and the oven was opened 4 times, and then the toluene mixture was freeze-dried for 48 hours to obtain the sample shown in FIG. 2.

### Embodiment 12

1. 0.5 g 8000 mesh flake graphite and 40 mL NMP (N-methylpyrrolidone) were added into a ball mill tank comprising zirconia milling balls and was planetary ball milled for 48 hours to obtain a mixture.

2. The mixture was filtered and washed by suction 10 times by distilled water and ethanol and was dried at 80° C. to obtain graphene.

3. 0.05 g industrial graphene and 4 mL 2-thiophene methanol was added into a three-necked flask to obtain a third solution.

4. The third solution was placed in oil bath maintained at 150° C. and was stirred by magnet, air in the three-necked flask was replaced by nitrogen at standard atmospheric pressure, and the third solution was reacted for 7 days.

5. 20 mL toluene and 100 mL deionized water were added into the third solution, and the deionized water was removed. 100 mL deionized water was added and removed 3 times to obtain toluene mixture.

6. The toluene mixture was dried (45° C., vacuum pressure) for 6 hours and the oven was opened 4 times, and then the toluene mixture was freeze-dried for 48 hours to obtain the sample shown in FIG. 2.

### Embodiment 13

1. 2 g 8000 mesh graphite powder, 12 g potassium permanganate, and 100 mL concentrated sulfuric acid was weighed.

2. 2 g graphite powder and 100 mL concentrated sulfuric acid were added into a 500 mL beaker, potassium permanganate was added into the 500 mL beaker in an ice bath to obtain a first solution, the first solution was kept in the ice bath for half an hour and was maintained at 35° C. for 2 hours, 150 mL distilled water was then slowly added into the first solution, and hydrogen peroxide was added into the first solution at room temperature.

3. The first solution was filtered by suction.

4. The first solution was respectively centrifuged with 5% dilute hydrochloric acid and distilled water.

5. The first solution was freeze-dried to obtain GO (graphene oxide)

6. 0.05 g GO was taken and evenly dispersed in 300 mL deionized water to obtain a second solution by high-frequency ultrasound, and 15 g VC (ascorbic acid) was taken and dissolved in the second solution.

7. The second solution was evenly dispersed and then was put in an oven at 80° C. for 4 hours.

8. The second solution was cooled to room temperature, filtered, washed by deionized water until residual material was washed away, and was dried in vacuum to obtain RGO.

9. 0.05 g RGO and 4 mL 2-furan methylamine was added into a three-necked flask to obtain a third solution.

10. The third solution was placed in oil bath maintained at 80° C. and was stirred by magnet, air in the three-necked flask was replaced by nitrogen at standard atmospheric pressure, and the third solution was reacted for 7 days.

11. 20 mL acetone and 120 mL deionized water were added into the third solution and the third solution was centrifugated for 0.5 hours to remove liquid of the third solution 3 times. A toluene mixture was obtained.

12. The toluene mixture was dried (45° C., vacuum pressure) for 6 hours and the oven was opened 4 times, and then the toluene mixture was freeze-dried for 48 hours to obtain the sample shown in FIG. 2.

### Embodiment 14

1. 2 g 8000 mesh graphite powder, 12 g potassium permanganate, and 100 mL concentrated sulfuric acid was weighed.

2. 2 g graphite powder and 100 mL concentrated sulfuric acid were added into a 500 mL beaker, potassium permanganate was added into the 500 mL beaker in an ice bath to obtain a first solution, the first solution was kept in the ice bath for half an hour and was maintained at 35° C. for 2 hours, 150 mL distilled water was then slowly added into the first solution, and hydrogen peroxide was added into the first solution at room temperature.

3. The first solution was filtered by suction.

4. The first solution was respectively centrifuged with 5% dilute hydrochloric acid and distilled water.

5. The first solution was freeze-dried to obtain GO (graphene oxide)

6. 0.05 g GO was taken and evenly dispersed in 300 mL deionized water to obtain a second solution by high-frequency ultrasound, and 15 g VC (ascorbic acid) was taken and dissolved in the second solution.

7. The second solution was evenly dispersed and then was put in an oven at 80° C. for 4 hours.

8. The second solution was cooled to room temperature, filtered, washed by deionized water until residual material was washed away, and was dried in vacuum to obtain RGO.

9. 0.05 g RGO and 4 mL 2-furan methanol was added into a three-necked flask to obtain a third solution.

10. The third solution was placed in oil bath maintained at 130° C. and was stirred by magnet, air in the three-necked flask was replaced by nitrogen at standard atmospheric pressure, and the third solution was reacted for 7 days.

11. 20 mL acetone and 120 mL deionized water were added into the third solution and the third solution was centrifugated for 0.5 hours to remove liquid of the third solution 3 times. A toluene mixture was obtained.

12. The toluene mixture was dried (45° C., vacuum pressure) for 6 hours and the oven was opened 4 times, and then the toluene mixture was freeze-dried for 48 hours to obtain the sample shown in FIG. 2.

### Embodiment 15

1. 2 g 8000 mesh graphite powder, 12 g potassium permanganate, and 100 mL concentrated sulfuric acid was weighed.

2. 2 g graphite powder and 100 mL concentrated sulfuric acid were added into a 500 mL beaker, potassium permanganate was added into the 500 mL beaker in an ice bath to obtain a first solution, the first solution was kept in the ice bath for half an hour and was maintained at 35° C. for 2 hours, 150 mL distilled water was then slowly added into the first solution, and hydrogen peroxide was added into the first solution at room temperature.

3. The first solution was filtered by suction.

4. The first solution was respectively centrifuged with 5% dilute hydrochloric acid and distilled water.

5. The first solution was freeze-dried to obtain GO (graphene oxide)

6. 0.05 g GO was taken and evenly dispersed in 300 mL deionized water to obtain a second solution by high-frequency ultrasound, and 15 g VC (ascorbic acid) was taken and dissolved in the second solution.

7. The second solution was evenly dispersed and then was put in an oven at 80° C. for 4 hours.

8. The second solution was cooled to room temperature, filtered, washed by deionized water until residual material was washed away, and was dried in vacuum to obtain RGO.

9. 0.05 g RGO and 4 mL 2-thiophene methylamine was added into a three-necked flask to obtain a third solution.

10. The third solution was placed in oil bath maintained at 80° C. and was stirred by magnet, air in the three-necked flask was replaced by nitrogen at standard atmospheric pressure, and the third solution was reacted for 7 days.

11. 20 mL acetone and 120 mL deionized water were added into the third solution and the third solution was centrifugated for 0.5 hours to remove liquid of the third solution 3 times. A toluene mixture was obtained.

12. The toluene mixture was dried (45° C., vacuum pressure) for 6 hours and the oven was opened 4 times, and then the toluene mixture was freeze-dried for 48 hours to obtain the sample shown in FIG. 2.

### Embodiment 16

1. 2 g 8000 mesh graphite powder, 12 g potassium permanganate, and 100 mL concentrated sulfuric acid was weighed.

2. 2 g graphite powder and 100 mL concentrated sulfuric acid were added into a 500 mL beaker, potassium permanganate was added into the 500 mL beaker in an ice bath to obtain a first solution, the first solution was kept in the ice bath for half an hour and was maintained at 35° C. for 2 hours, 150 mL distilled water was then slowly added into the first solution, and hydrogen peroxide was added into the first solution at room temperature.

3. The first solution was filtered by suction.

4. The first solution was respectively centrifuged with 5% dilute hydrochloric acid and distilled water.

5. The first solution was freeze-dried to obtain GO (graphene oxide)

6. 0.05 g GO was taken and evenly dispersed in 300 mL deionized water to obtain a second solution by high-frequency ultrasound, and 15 g VC (ascorbic acid) was taken and dissolved in the second solution.

7. The second solution was evenly dispersed and then was put in an oven at 80° C. for 4 hours.

8. The second solution was cooled to room temperature, filtered, washed by deionized water until residual material was washed away, and was dried in vacuum to obtain RGO.

9. 0.05 g RGO and 4 mL 2-thiophene methanol was added into a three-necked flask to obtain a third solution.

10. The third solution was placed in oil bath maintained at 150° C. and was stirred by magnet, air in the three-necked flask was replaced by nitrogen at standard atmospheric pressure, and the third solution was reacted for 7 days.

11. 20 mL acetone and 120 mL deionized water were added into the third solution and the third solution was centrifugated for 0.5 hours to remove liquid of the third solution 3 times. A toluene mixture was obtained.

12. The toluene mixture was dried (45° C., vacuum pressure) for 6 hours and the oven was opened 4 times, and then the toluene mixture was freeze-dried for 48 hours to obtain the sample shown in FIG. 2.

### Embodiment 17

1. 0.05 g G2 (industrial graphene obtained by a mechanical stripping method, that is, industrial mechanical stripping graphene) and 4 mL 2-furan methylamine was added into a three-necked flask to obtain a third solution.

2. The third solution was placed in oil bath maintained at 80° C. and was stirred by magnet, air in the three-necked flask was replaced by nitrogen at standard atmospheric pressure, and the third solution was reacted for 7 days.

3. 20 mL acetone and 120 mL deionized water were added into the third solution and the third solution was centrifugated for 0.5 hours to remove liquid of the third solution 3 times. A toluene mixture was obtained.

4. The toluene mixture was dried (45° C., vacuum pressure) for 6 hours and the oven was opened 4 times, and then the toluene mixture was freeze-dried for 48 hours to obtain the sample shown in FIG. 2.

### Embodiment 18

1. 0.05 g G2 (industrial graphene obtained by a mechanical stripping method) and 4 mL 2-furan methanol was added into a three-necked flask to obtain a third solution.

2. The third solution was placed in oil bath maintained at 130° C. and was stirred by magnet, air in the three-necked flask was replaced by nitrogen at standard atmospheric pressure, and the third solution was reacted for 7 days.

3. 20 mL acetone and 120 mL deionized water were added into the third solution and the third solution was centrifugated for 0.5 hours to remove liquid of the third solution 3 times. A toluene mixture was obtained.

4. The toluene mixture was dried (45° C., vacuum pressure) for 6 hours and the oven was opened 4 times, and then the toluene mixture was freeze-dried for 48 hours to obtain the sample shown in FIG. 2.

### Embodiment 19

1. 0.05 g G2 (industrial graphene obtained by a mechanical stripping method) and 4 mL 2-thiophene methylamine was added into a three-necked flask to obtain a third solution.

2. The third solution was placed in oil bath maintained at 80° C. and was stirred by magnet, air in the three-necked flask was replaced by nitrogen at standard atmospheric pressure, and the third solution was reacted for 7 days.

3. 20 mL acetone and 120 mL deionized water were added into the third solution and the third solution was centrifugated for 0.5 hours to remove liquid of the third solution 3 times. A toluene mixture was obtained.

4. The toluene mixture was dried (45° C., vacuum pressure) for 6 hours and the oven was opened 4 times, and then the toluene mixture was freeze-dried for 48 hours to obtain the sample shown in FIG. 2.

### Embodiment 20

1. 0.05 g G2 (industrial graphene obtained by a mechanical stripping method) and 4 mL 2-thiophene methanol was added into a three-necked flask to obtain a third solution.

2. The third solution was placed in oil bath maintained at 150° C. and was stirred by magnet, air in the three-necked flask was replaced by nitrogen at standard atmospheric pressure, and the third solution was reacted for 7 days.

3. 20 mL acetone and 120 mL deionized water were added into the third solution and the third solution was centrifugated for 0.5 hours to remove liquid of the third solution 3 times. A toluene mixture was obtained.

4. The toluene mixture was dried (45° C., vacuum pressure) for 6 hours and the oven was opened 4 times, and then the toluene mixture was freeze-dried for 48 hours to obtain the sample shown in FIG. 2.

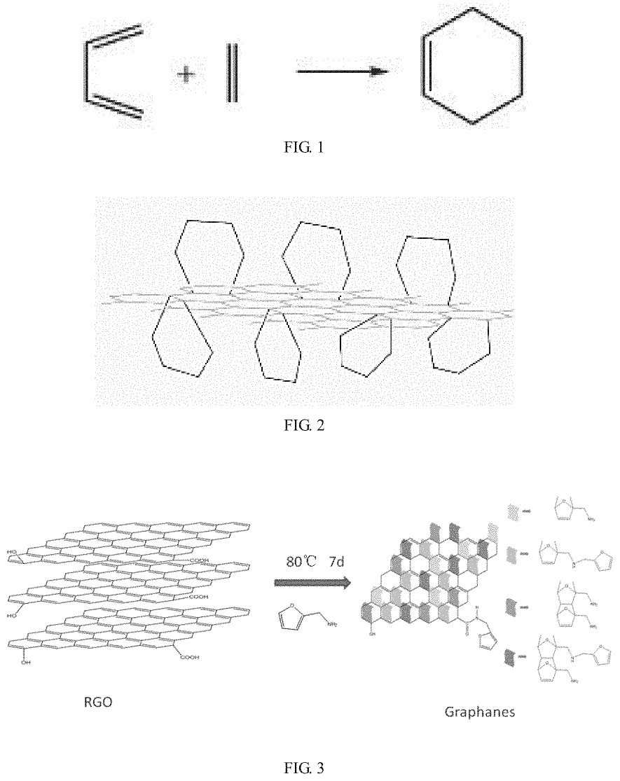

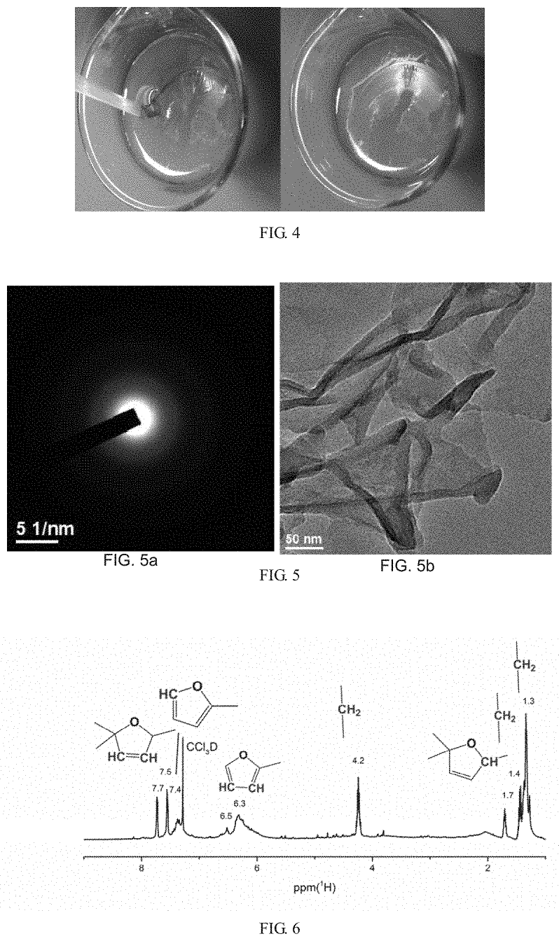

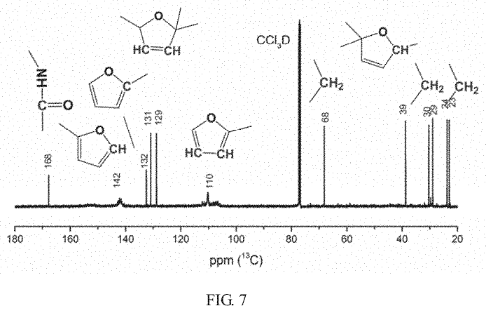

The specific reaction principles of the embodiments are shown in FIGS. 1 and 3. Referring to FIG. 2, the final sample is a three-dimensional macromolecular structure formed by bonding organic molecules on planar six-membered carbon rings comprising continuous spa hybrids. Because the basic structure of the three-dimensional macromolecule is very different from the traditional linear molecular structure, the three-dimensional macromolecule will have many different properties from existing materials and have advantages in many properties. For example, the three-dimensional macromolecules obtained by the reaction between graphene and furan methylamine have good dissolution properties and can be dissolved in organic solvents such as ethanol, toluene, acetone, tetrahydrofuran, chloroform, cyclohexanone, etc., methyl methacrylate, styrene, polypolyacid, epoxy prepolymer, and other polymer monomers. The three-dimensional macromolecule can form a film, form a transparent film on a surface of water, has a certain toughness (see FIG. 4), and so on. The selected diffraction by TEM shows that the prepared product no longer has the original graphene diffraction ring (see FIG. 5), indicating that the regular two-dimensional crystal structure of the original graphene disappears and turns into a nonconjugated structure of graphane. The hydrogen nuclear magnetic resonance spectrum and carbon nuclear magnetic resonance spectrum show that the newly generated methylene and methine groups of sp³C appear on the prepared product (see FIGS. 6 and 7), indicating that 2-furan methylamine is grafted onto the planar six-membered carbon rings comprising the continuous sp³ hybrids by the D-A reaction.

The aforementioned embodiments are merely some embodiments of the present disclosure, and the scope of the disclosure is not limited thereto. Thus, it is intended that the present disclosure cover any modifications and variations of the presently presented embodiments provided they are made without departing from the appended claims and the specification of the present disclosure.

What is claimed is:

1. A substituted graphane material with a three-dimensional structure, comprising:
a planar substrate with a plurality of six-membered carbon rings comprising continuous sp³ hybrids, wherein an organic molecular ring is connected to the planar substrate due to a Diels-Alder (D-A) reaction.

2. The substituted graphane material according to claim 1, wherein the organic molecular ring is a ring merely comprising a single organic molecular or a ring comprising a plurality of continuous organic molecules.

3. The substituted graphane material according to claim 1, wherein the organic molecular ring is distributed on a side of a plane, and the planar substrate is disposed on the plane.

4. The substituted graphane material according to claim 1, wherein the organic molecular ring is distributed on two sides of a plane, and the planar substrate is disposed on the plane.

5. A method for preparing the substituted graphane material according to claim 1, comprising:
(1) mixing graphene material and conjugated diene, heating in inert gas or nitrogen atmosphere to a temperature at which the conjugated diene is configured to form a reflux, and refluxing and reacting for 6-8 days to obtain a material; and
(2) removing impurities of the material with a solvent and drying to remove the solvent such that the substituted graphane material is obtained.

6. The method according to claim 5, wherein the conjugated diene comprises 2-furan methylamine, 2-thienyl methylamine, 2-furan methanol, or 2-thienyl methanol.

7. The method according to claim 5, comprising:
preparing graphene oxide, comprising:
weighing graphite powder, potassium permanganate, and concentrated sulfuric acid according to a preset ratio;
adding the graphite powder and the concentrated sulfuric acid to a container;
adding the potassium permanganate into the container in ice bath;
maintaining the container in the ice bath for half an hour;
maintaining the container at 35° C. for 2 hours and then adding 150 mL distilled water;
cooling to room temperature and then adding hydrogen peroxide;
filtering by suction;
centrifuging with dilute hydrochloric acid and distilled water respectively; and
freeze-drying to obtain the graphene oxide.

8. The method according to claim 7, comprising:
dissolving the graphene oxide in an ascorbic acid solution, uniformly dispersing, and drying to obtain the graphene material, wherein the graphene material is a reduced graphene oxide.

9. The method according to claim 5, comprising:
adding flake graphite and N-methylpyrrolidone into a ball mill tank with zirconia milling balls;
planetary ball milling for 48 hours;
filtering and washing by distilled water and ethanol respectively; and
drying at 80° C. to obtain the graphene material.

10. The method according to claim 6, comprising:
preparing graphene oxide, comprising:
weighing graphite powder, potassium permanganate, and concentrated sulfuric acid according to a preset ratio;
adding the graphite powder and the concentrated sulfuric acid to a container;
adding the potassium permanganate into the container in ice bath;
maintaining the container in the ice bath for half an hour;
maintaining the container at 35° C. for 2 hours and then adding 150 mL distilled water;
cooling to room temperature and then adding hydrogen peroxide;
filtering by suction;
centrifuging with dilute hydrochloric acid and distilled water respectively; and
freeze-drying to obtain the graphene oxide.

11. The method according to claim 10, comprising:
dissolving the graphene oxide in an ascorbic acid solution, uniformly dispersing, and drying to obtain the graphene material, wherein the graphene material is a reduced graphene oxide.

12. The method according to claim 6, comprising:
adding flake graphite and N-methylpyrrolidone into a ball mill tank with zirconia milling balls;
planetary ball milling for 48 hours;
filtering and washing by distilled water and ethanol respectively; and
drying at 80° C. to obtain the graphene material.

13. The method according to claim 5, wherein the solvent comprises at least one of water, toluene, acetone, or chloroform.

14. The method according to claim 5, wherein the inert gas is argon gas.

15. The method according to claim 5, wherein the graphene material is reduced graphene oxide or mechanical stripping graphene.

16. The method according to claim 5, wherein a temperature of the reflux is 80° C.-150° C.
